(19) United States
(12) Patent Application Publication
(10) Pub. No.: US 2024/0296203 A1
(43) Pub. Date: Sep. 5, 2024

(54) DATA PROCESSING METHOD, DATA PROCESSING APPARATUS, AND NON-TRANSITORY COMPUTER-READABLE STORAGE MEDIUM

(71) Applicant: SHIMADZU CORPORATION, Kyoto-shi (JP)

(72) Inventors: Satoshi SHIMIZU, Kyoto-shi (JP); Shiori NAGAI, Kyoto-shi (JP); Kenta ADACHI, Kyoto-shi (JP)

(21) Appl. No.: 18/418,004

(22) Filed: Jan. 19, 2024

(30) Foreign Application Priority Data
Mar. 1, 2023 (JP) 2023-031161

(51) Int. Cl. G06F 17/18

(52) U.S. Cl. CPC G06F 17/18

(57) ABSTRACT

A data processing method according to one aspect of the present invention includes: a step of collecting an analysis file set including analysis data by an analyzer from a plurality of types of analyzers; a step of selecting a plurality of analysis file sets to be analyzed from the plurality of types of collected analysis file sets according to a narrowing-down condition received from a user; a step of extracting a plurality of features from each of the plurality of selected analysis file sets; a step of summarizing the plurality of features by performing statistical processing of the plurality of features; and a step of performing an analysis using the summarized features. The step of summarizing the plurality of features includes a step of presenting statistical information generated in the statistical processing to the user.

### CROSS-REFERENCE TO RELATED APPLICATIONS

This application claims priority under 35 U.S.C. § 119 to Japanese Patent Application No. 2023-31161 filed on Mar. 1, 2023, the entire disclosure of which is incorporated herein by reference in its entirety.

### BACKGROUND OF THE INVENTION

### Field of the Invention

The present invention relates to a data processing method, a data processing apparatus, and a non-transitory computer-readable storage medium, and more specifically to a data processing method for processing analysis data acquired by a plurality of types of analyzers, a data processing apparatus, and a non-transitory computer-readable storage medium.

### Description of the Related Art

The following description sets forth the inventor's knowledge of the related art and problems therein and should not be construed as an admission of knowledge in the prior art.

International Publication No. WO 2021/235111 discloses a system for analyzing analysis data acquired by a plurality of analyzers cross-sectionally. In this system, the analysis data acquired by the plurality of types of analyzers are stored in a database. The data processing apparatus analyzes the analysis data stored in the database using dedicated data analysis software to generate features for use in statistical analysis or AI analysis. The data processing apparatus then performs machine learning based on the generated features to construct a trained model.

Because the features acquired from a plurality of types of analyzers vary widely in types, the user must select, from among the plurality of types of features stored in the database, features suitable for use in statistical analysis or AI analysis.

However, in the case where a plurality of analysis data of the same type is acquired for one material, a plurality of features of the same type will be generated from the plurality of analysis data. In such a case, the user is required to perform statistical processing, such as, e.g., averaging, to summarize the plurality of features.

However, when the plurality of features includes outliers, a problem occurs that the plurality of features cannot be appropriately summarized by averaging processing. For this reason, the user is required to confirm the distribution state of the plurality of features, and therefore, there is a concern that a great deal of time and effort will be required to acquire appropriate features.

### SUMMARY OF THE INVENTION

The present disclosure has been made to solve such problems, and the purpose of the present disclosure is to provide a user interface for supporting a task of summarizing a plurality of features acquired from analysis data acquired by a plurality of types of analyzers.

A data processing method according to one aspect of the present invention includes:
a step of collecting an analysis file set including analysis data by an analyzer from a plurality of types of analyzers;
a step of selecting a plurality of analysis file sets to be analyzed from the plurality of types of collected analysis file sets according to a narrowing-down condition received from a user;
a step of extracting a plurality of features from each of the plurality of selected analysis file sets;
a step of summarizing the plurality of features by performing statistical processing of the plurality of features; and
a step of performing an analysis using the summarized features.

The step of summarizing the plurality of features includes a step of presenting statistical information generated in the statistical processing to the user.

The data processing apparatus according to a second aspect of the present invention is a data processing apparatus capable of communicating with a plurality of types of analyzers, the data processing apparatus comprising:
a processor; and
a memory configured to store a program to be executed by the processor.

The processor is configured to, in accordance with the program,
collect an analysis file set including analysis data by an analyzer from the plurality of types of analyzers,
select a plurality of analysis file sets to be analyzed from the plurality of types of collected analysis file sets according to a narrowing-down condition received from a user,
extract a plurality of features from each of the plurality of selected analysis file sets,
summarize the plurality of features by performing statistical processing of the plurality of features, and
perform an analysis using the summarized features.

The processor presents statistical information generated in the statistical processing to the user when summarizing the plurality of features.

A non-transitory computer-readable storage medium according to a third aspect of the present invention stores a program to be executed by a computer.

The program makes a computer execute:
a step of collecting an analysis file set including analysis data by an analyzer from a plurality of types of analyzers;
a step of selecting a plurality of analysis file sets to be analyzed from the plurality of types of collected analysis file sets according to a narrowing-down condition received from a user;
a step of extracting a plurality of features from each of the plurality of selected analysis file sets;
a step of summarizing the plurality of features by performing statistical processing of the plurality of features; and
a step of performing an analysis using the summarized features.

The step of summarizing the plurality of features includes a step of presenting statistical information generated in the statistical processing to the user.

The above and other objects, features, aspects and advantages of the present invention will become apparent from the following detailed description of this invention understood in connection with the accompanying drawings.

### BRIEF DESCRIPTION OF THE DRAWINGS

The preferred embodiments of the present invention are shown by way of example, and not limitation, in the accompanying figures.

FIG. 1 is a schematic diagram for explaining a configuration example of an analysis system according to an embodiment of the present invention.

FIG. 2 is a schematic diagram of a hardware configuration example of an information processing apparatus and a data processing apparatus.

FIG. 3 is a diagram schematically showing a functional configuration of an information processing apparatus and a data processing apparatus.

FIG. 4 is a diagram showing a configuration example of an analysis file DB.

FIG. 5 is a diagram showing one example of a feature table.

FIG. 6 is a flowchart for explaining processing steps performed by a data processing apparatus.

FIG. 7 is a view schematically showing a first example of a UI screen displayed on a display unit.

FIG. 8 is a view schematically showing a first example of a UI screen displayed on a display unit.

FIG. 9 is a view schematically showing a first example of a UI screen displayed on a display unit.

FIG. 10 is a view schematically showing a second example of a UI screen displayed on a display unit.

FIG. 11 is a view schematically showing a third example of a UI screen displayed on a display unit.

FIG. 12 is a view schematically showing a fourth example of a UI screen displayed on a display unit.

FIG. 13 is a diagram showing an example of a table generated by a data processing apparatus.

### DETAILED DESCRIPTION OF THE PREFERRED EMBODIMENTS

Hereinafter, some embodiments of the present invention will be described with reference to the attached drawings. Note that, hereinafter, the same or equivalent portion in the figures is assigned by the same reference symbol, and the description thereof will not be repeated.

### <Configuration Example of Analysis System>

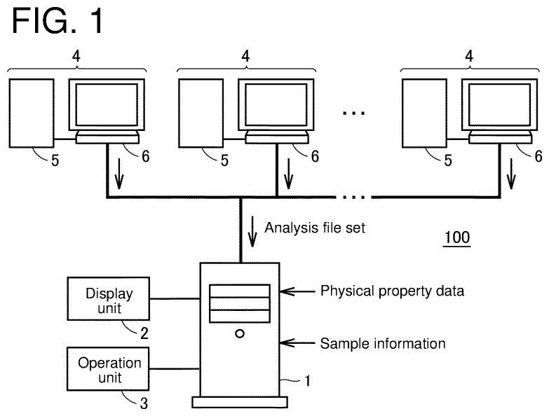

FIG. 1 is a schematic diagram for describing a configuration example of an analysis system according to an embodiment of the present invention. The analysis system according to this embodiment can be applied to a system for analyzing analysis data acquired by a plurality of analyzers cross-sectionally. As shown in FIG. 1, this analysis system 100 is equipped with a plurality of analyzers 4 and a data processing apparatus 1.

The plurality of analyzers 4 each measures a sample. The plurality of analyzers 4 includes a plurality of types of analyzers. In one aspect, the plurality of analyzers 4 includes, for example, a liquid chromatograph (LC), a gas chromatograph (GC), a liquid chromatograph mass spectrometer (LC-MS), a gas chromatograph mass spectrometer (GC-MS), a pyrolysis gas chromatograph mass spectrometry (Py-GC/MS), a scanning electron microscope (SEM), a transmission electron microscope (TEM), an energy dispersive X-ray fluorescence analyzer (EDX), a wavelength dispersive fluorescent X-ray analyzer (WDX), a nuclear magnetic resonator (NMR), and a Fourier transform infrared spectrophotometer (FT-IR).

The plurality of analyzers 4 may further include, for example, a photodiode array detector (LC-PDA), a liquid chromatograph tandem mass spectrometer (LC/MS/MS), a gas chromatograph tandem mass spectrometer (GC/MS/MS), a liquid chromatograph ion trap time-of-flight mass spectrometer (LC/MS-IT-TOF), a near-infrared spectrometer, a tensile tester, a compression testing machine, an emission spectroscopic analyzer (AES), an atomic absorption analyzer (AAS/FL-AAS), a plasma mass spectrometer (ICP-MS), an organic element analyzer, a glow discharge mass spectrometer (GDMS), a particle composition analyzer, a trace total nitrogen automatic analyzer (TN), a high-sensitivity nitrogen carbon analyzer (NC), and a thermal analyzer.

The analysis system 100 has a plurality of analyzers 4, which makes it possible to perform multifaceted analyses of a single sample using a plurality of types of analysis data.

The analyzer 4 includes a device body 5 and an information processing apparatus 6. The device body 5 measures a sample to be analyzed. To the information processing apparatus 6, sample identification information, sample measurement conditions, etc., are input.

The information processing apparatus 6 controls the measurement in the device body 5 according to the input measurement conditions. With this, analysis data based on the measurement results of the sample are acquired. The analysis data include, for example, electron microscope images acquired by an SEM or a TEM, chromatograms and mass spectra acquired by a GC-MS or an LC-MS, as well as spectra acquired from an FT-IR or an NMR.

The information processing apparatus 6 stores the acquired analysis data, along with the sample identification information and the sample measurement conditions, in a data file, and stores the data file in a built-in memory. In this specification, this data file is also referred to as “analysis file set.”

The information processing apparatus 6 is connected to the data processing apparatus 1 in a mutually communicable manner. The connection between the information processing apparatus 6 and the data processing apparatus 1 may be wired or wireless. For example, as a communication network connecting the information processing apparatus 6 and the data processing apparatus 1, the Internet can be used. With this, the information processing apparatus 6 of each analyzer 4 can transmit an analysis file set, which is a data file for each sample, to the data processing apparatus 1.

The data processing apparatus 1 is principally a device for managing the analysis data acquired by the plurality of analyzers 4. An analysis file set is input to the data processing apparatus 1 from each of the analyzers 4. It is possible to further input information on the sample (hereinafter also referred to as “sample information”) and physical property data of the sample to the data processing apparatus 1.

The sample information includes identification information for identifying the sample (the sample ID, the sample name, etc.) and information on the sample production (hereinafter also referred to as “recipe data”). The recipe data of a sample can include, for example, information on the blending quantities of sample raw materials and the sample production process. The physical property data of a sample is data indicating the sample's attributes acquired by means other than the analysis by the analyzer 4.

The data processing apparatus 1 is provided with a built-in database. The database is a storage unit for storing data exchanged between the data processing apparatus 1 and the plurality of analyzers 4, data input from outside the data processing apparatus 1, and data generated in the data processing apparatus 1. The data processing apparatus 1 stores the analysis file set, and the sample information and the sample's physical property data in a database for each sample, in a linked manner. Note that in the example shown in FIG. 1, it is configured such that the database is built in the data processing apparatus 1, but it can also be configured such that the database is externally attached to the data processing apparatus 1.

### <Hardware Configuration Example of Analysis System>

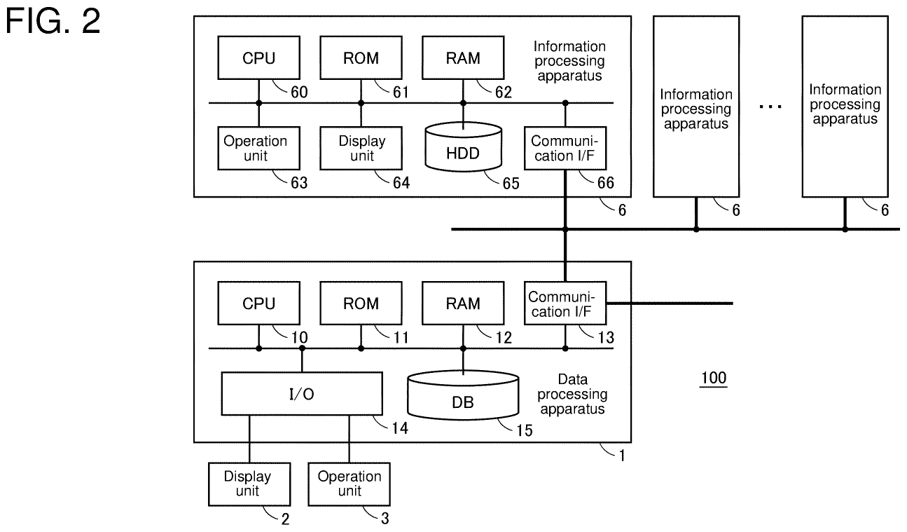

FIG. 2 is a diagram schematically showing a hardware configuration example of the information processing apparatus 6 and the data processing apparatus 1.

### (Hardware Configuration Example of Information Processing Apparatus 6)

As shown in FIG. 2, the information processing apparatus 6 is equipped with a CPU (Central Processing Unit) 60 for controlling the entire analyzer 4 and a storage unit for storing programs and data and is configured to operate according to a program.

The storage unit includes a ROM (Read Only Memory) 61, a RAM (Random Access Memory) 62, and an HDD (Hard Disk Drive) 65. The ROM 61 stores a program to be executed by the CPU 60. The RAM 62 temporarily stores data used during the execution of a program in the CPU 60. The RAM 62 serves as a temporary data memory used as a working area. The HDD 65 is a non-volatile storage device and stores information, such as, e.g., analysis file sets, generated by the information processing apparatus 6. In addition to the HDD 65 or instead of the HDD 65, a semiconductor memory device, such as, e.g., a flash memory, may be used.

The information processing apparatus 6 further includes a communication interface (I/F) 66, an operation unit 63, and a display unit 64. The communication I/F 66 is an interface for the information processing apparatus 6 to communicate with external devices including the device body 5 and the data processing apparatus 1.

The operation unit 63 receives an input including an instruction to the information processing apparatus 6 from the user. The operation unit 63 includes a keyboard, a mouse, and a touch panel integrally configured with the display screen of the display unit 64 to receive sample measurement conditions and sample identification information.

When setting measurement conditions, the display unit 64 can display, for example, an input screen for measurement conditions and sample identification information. During the measurement, the display unit 64 can display the measurement data detected by the device body 5 and the data analysis results by the information processing apparatus 6.

The processing by the analyzer 4 is realized by hardware and software executed by the CPU 60. In some cases, such software is stored in advance in the ROM 61 or the HDD 65. Further, there is a case in which software is distributed as a program product stored in a storage medium, which is not shown in the figure. The software is then read out from the HDD 65 by the CPU 60 and stored in the RAM 62 in a format executable by the CPU 60. The CPU 60 executes this program.

### (Hardware Configuration of Data Processing Apparatus 1)

The data processing apparatus 1 is equipped with a CPU 10 to control the entire apparatus and storage units for storing programs and data and is configured to operate according to a program. The storage units include a ROM 11, a RAM 12, and a database (DB) 15.

The ROM 11 stores programs to be executed by the CPU 10. The RAM 12 temporarily stores data used during the execution of a program in the CPU 10. The RAM 12 functions as a temporary data memory used as a working area.

The DB 15 is a nonvolatile storage device, and stores data exchanged between the data processing apparatus 1 and the plurality of analyzers 4, data input from outside the data processing apparatus 1, and data generated in the data processing apparatus 1. The DB 15, as will be described later, is configured to include an analysis file DB 15A for storing analysis file sets collected from the plurality of analyzers 4, a statistical information DB 15B for storing statistical information obtained by statistical processing of the plurality of analysis file sets, and a feature DB 15C for storing information on the features acquired from the plurality of analysis file sets.

The data processing apparatus 1 further includes a communication I/F 13 and an input/output interface (I/O) 14. The communication I/F 13 is an interface for the data processing apparatus 1 to communicate with external devices including the information processing apparatus 6.

The I/O 14 is an interface for inputs to or outputs from the data processing apparatus 1. The I/O 14 is connected to a display unit 2 and an operation unit 3. When executing the processing to generate a feature table from a plurality of analysis file sets, in the data processing apparatus 1, the display unit 2 can display information on the processing and a user interface screen for receiving user operations.

The operation unit 3 receives inputs including user instructions. The operation unit 3 includes a keyboard and a mouse and receives sample information and sample physical property data. Note that it may be configured such that the sample information and the sample physical property data are received from an external device via the communication I/F 13.

### <Functional Configuration of Analysis System>

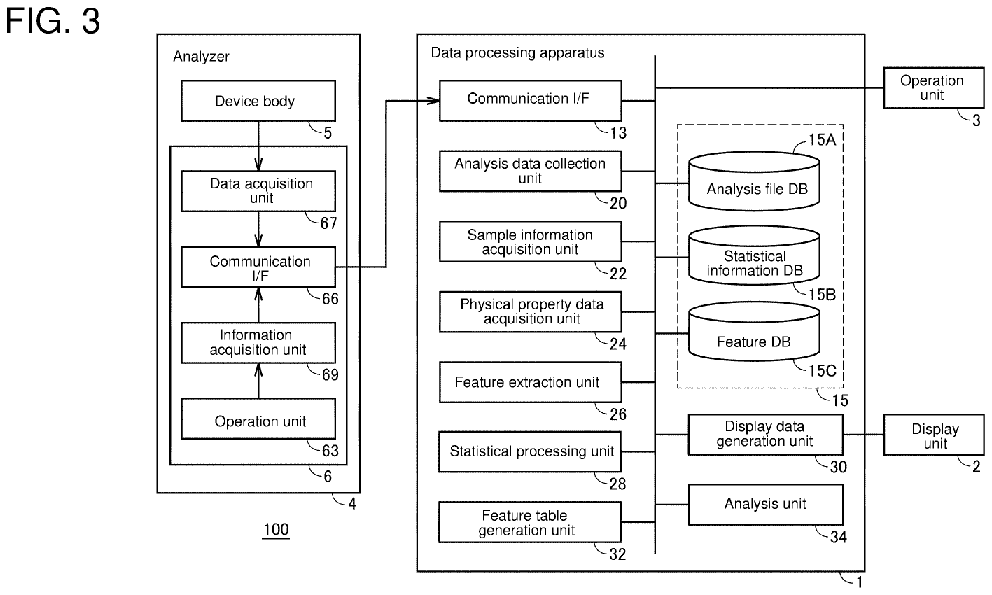

FIG. 3 is a diagram schematically showing a functional configuration of the information processing apparatus 6 and the data processing apparatus 1.

### (Functional Configuration of Information Processing Apparatus 6)

As shown in FIG. 3, the information processing apparatus 6 is configured to include a data acquisition unit 67 and an information acquisition unit 69. These functional configurations are realized by the CPU 60 executing a predetermined program in the information processing apparatus 6 shown in FIG. 2.

The data acquisition unit 67 acquires the analysis data based on the measurement results of the sample from the device body 5. For example, in the case where the analyzer 4 is a GC-MS, the analysis data include chromatograms and mass spectra. In the case where the analyzer 4 is an SEM or a TEM, the analysis data include image data showing the electron microscope image of the sample. The data acquisition unit 67 transfers the acquired analysis data to the communication I/F 66.

The information acquisition unit 69 acquires the information received by the operation unit 63. Specifically, the information acquisition unit 69 acquires sample identification information and information indicating measurement conditions of the sample. The sample identification information includes, for example, a sample name, a product name, a model number, and a serial number of the product to be sampled. The sample measurement conditions include device parameters including a name and a model number of the analyzer to be used, and measurement parameters indicating measurement conditions, such as, e.g., voltage and/or current application conditions and temperature conditions.

The communication I/F 66 transmits an analysis file set in which the acquired analysis data, measurement conditions, and sample identification information are combined into one file set to the data processing apparatus 1.

### (Functional Configuration of Data Processing Apparatus 1)

The data processing apparatus 1 is equipped with an analysis data collection unit 20, a sample information acquisition unit 22, a physical property data acquisition unit 24, a feature extraction unit 26, a statistical processing unit 28, a display data generation unit 30, a feature table generation unit 32, an analysis unit 34, an analysis file database (DB) 15A, a statistical information DB 15B and a features DB 15C. These functional configurations are realized by the CPU 10 executing a predetermined program in the data processing apparatus 1 shown in FIG. 2.

The analysis data collection unit 20 collects an analysis file set transmitted from the information processing apparatus 6 of each analyzer 4 via the communication I/F 13. The analysis file set includes the sample analysis data, the sample identification information, and the measurement conditions. The analysis data collection unit 20 stores the collected analysis file sets in the analysis file DB 15A. The analysis file DB 15A stores a wide variety of analysis file sets collected from the plurality of analyzers 4.

The sample information acquisition unit 22 acquires the sample information received by the operation unit 3. The sample information includes the sample identification information (sample ID, sample name, etc.) and the sample recipe data.

The physical property data acquisition unit 24 acquires the physical property data of the sample received by the operation unit 3. The physical property data of the sample is data indicating the sample's attributes, including, for example, a value indicating the performance of the sample and a value indicating the deterioration degree of the sample.

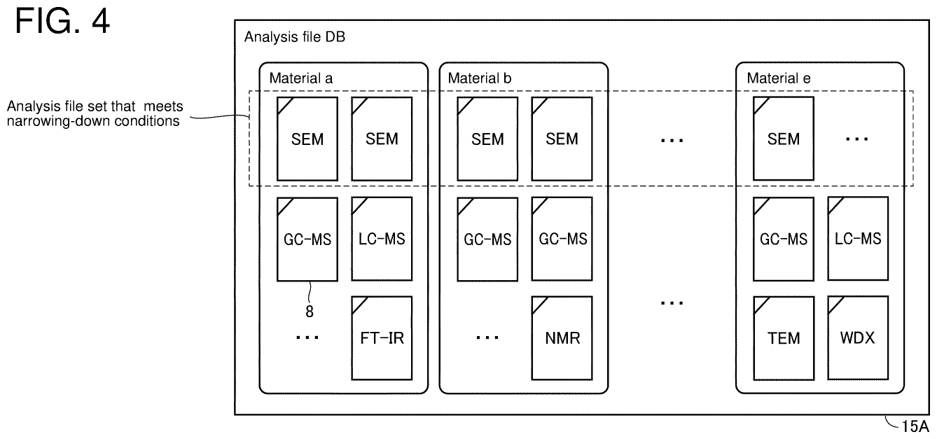

In the analysis file DB 15A, the analysis file sets collected by the analysis data collection unit 20, and the sample information and the physical property data acquired by the sample information acquisition unit 22 and the physical property data acquisition unit 24 are saved in a linked manner. FIG. 4 is a diagram showing a configuration example of the analysis file DB 15A. As shown in FIG. 4, a plurality of analysis file sets 8 are sorted and saved by each material in the analysis file DB 15A. Which material each analysis file set 8 is sorted into can be determined from the sample identification information included in the analysis file set 8 or from the sample information linked to the analysis file set 8. In this way, for a single material, a plurality of analysis file sets 8 collected from a plurality of types of analyzers 4 is stored.

Returning to FIG. 3, the feature extraction unit 26 generates features by analyzing each analysis file set stored in the analysis file DB 15A using dedicated data analysis software. The feature data include, for example, electron microscope images acquired by an SEM or a TEM, chromatograms acquired by a GC-MS or an LC-MS, analysis data such as spectra acquired by an FT-IR or an NMR, as well as a sample composition, a concentration, a molecular structure, the number of molecules, a molecular weight, a degree of polymerization, a particle diameter, a particle area, the number of particles, a particle dispersion, a peak intensity, peak area, a peak slope, a compound concentration, a compound amount, an absorbance, a reflectance, a transmittance, a sample test intensity, a Young's modulus, a tensile strength, a deformation amount, a strain amount, a fracture time, an average interparticle distance, a dielectric dissipation factor, an elongation, a spring strength, a loss factor, a glass dislocation temperature, and a thermal expansion coefficient, which are calculated from the analysis data.

The feature extraction unit 26 stores the generated features in the feature DB 15C by linking them to the analysis file set. Note that in the example shown in FIG. 3, the feature extraction unit 26 is included in the data processing apparatus 1 but is not limited thereto. The information processing apparatus 6 of each analyzer 4 may have a feature extraction unit.

The statistical processing unit 28 acquires narrowing-down conditions received by the operation unit 3. In this specification, the “narrowing-down conditions” are conditions for narrowing down the analysis file sets to be targeted for a statistical analysis or an AI (Artificial Intelligence) analysis, etc., from a wide variety of analysis file sets stored in the analysis file DB 15A. The narrowing-down conditions include at least the type of the analyzer.

The statistical processing unit 28 narrows down the analysis file set that meets the narrowing-down conditions by selecting from the wide variety of analysis file sets stored in the analysis file DB 15A (see FIG. 4).

The statistical processing unit 28 further acquires the visualization conditions received by the operation unit 3. In this specification, the “visualization conditions” are conditions for visualizing the distribution state of the plurality of features extracted from the plurality of analysis file sets that meet the narrowing-down conditions. The visualization conditions include conditions for grouping the plurality of features extracted from the plurality of analysis file sets and conditions for graphing the distribution state of the plurality of grouping conditions.

In grouping, the plurality of features extracted from the plurality of analysis file sets can be categorized by material or by analysis file set. For example, in the case of grouping by material, features extracted from at least one analysis file set belong to each group per one material. On the other hand, in the case of grouping by analysis file set, features extracted from a single analysis file set belong to each group. Note that it is also possible to choose not to perform grouping.

In grouping, a statistic which is a numerical value summarizing the characteristics of the features categorized in each group is further calculated. This statistic is referred to as a summary statistic. The statistic includes, for example, an average value, a median value, a mode value, a maximum value, a minimum value, a standard deviation, a variance, a skewness, and a kurtosis. Among these statistics, an average value, a median value, a mode value, a maximum value, and a minimum value correspond to representative values that represent the entirety of the features classified into each group. A standard deviation, a variance, a skewness, and a kurtosis correspond to a dispersion that represents the dispersion of the features in each group.

Graphing is expressing, in a graphical form, the distribution state of statistics for the statistics of the features of the plurality of groups acquired by grouping. The user can set the type of the graph and the coordinate axes ((the horizontal axis (X-axis) and the vertical axis (Y-axis)) of the graph as graphing conditions. The details of the graph are described below.

The statistical processing unit 28 performs statistical processing (grouping and graphing) of the plurality of analysis file sets according to the visualization conditions, thereby generating statistical information representing the distribution status of the plurality of features to be extracted from the plurality of analysis file sets. The statistical processing unit 28 stores the generated statistical information in the statistical information DB 15B in response to the user's input operations to the operation unit 3. In the statistical information DB 15B, the information on a plurality of narrowed-down file sets and the statistical information on the plurality of file sets are saved in a linked manner.

The display data generation unit 30 generates data to display the statistical information generated by the statistical processing unit 28 on the display screen of the display unit 2. The display data generation unit 30 displays a graph representing the distribution state of the statistics of the plurality of feature values for the plurality of groups on the display unit 2. By referring to the graph, the user can visually confirm the distribution state of the features of the plurality of groups. This allows the user to determine how the plurality of analysis file sets should be grouped together in a feature table.

The display data generation unit 30 further displays a user interface (UI) screen to receive user operations regarding narrowing-down conditions and visualization conditions to be given to the statistical processing unit 28 and user instructions to be given to the feature table generation unit 32.

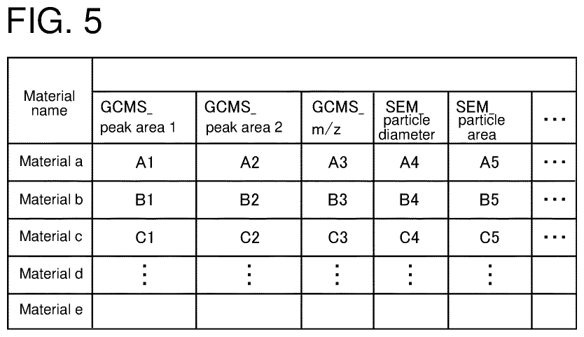

The feature table generation unit 32 generates a feature table in which the features are recorded. In this specification, the “feature table” is a table format that summarizes the features used for analyses when performing statistical and/or AI analyses of the plurality of analysis file sets. FIG. 5 is a diagram showing one example of a feature table. In the example shown in FIG. 5, the feature table records, for each material, a plurality of types of features extracted from the plurality of analysis file sets belonging to the material. The feature table can also record, in addition to the features, the calculated value obtained by performing arithmetic processing on the physical properties of the sample or at least one of the features.

The feature table generation unit 32 records the statistics of the features classified into each group in the feature table, which are calculated by the grouping in the statistical processing unit 28, in response to the user's input operation to the operation unit 3. For example, in the case where a plurality of features extracted from a plurality of analysis file sets is grouped by material, the feature table records the statistics of the features for each material, as shown in FIG. 5.

Further, although not shown in the figures, in the case where a plurality of features extracted from a plurality of analysis file sets is grouped for each analysis file set, the feature table records the statistics of the features for each analysis file set. The feature table generated in this way is stored in the feature DB 15C by being linked to the information on the narrowed-down analysis file sets.

The analysis unit 34 performs an AI analysis and a statistical analysis, etc., using the feature table saved in the feature DB 15C. The method of machine learning in the analysis unit 34 is not particularly limited, and for example, known machine learning, such as, e.g., neural networks (NN: Neural Network) and support vector machines (SVM: Support Vector Machine) can be used.

### <Operation of Data Processing Apparatus 1>

Next, the processing performed by the data processing apparatus 1 will be described.

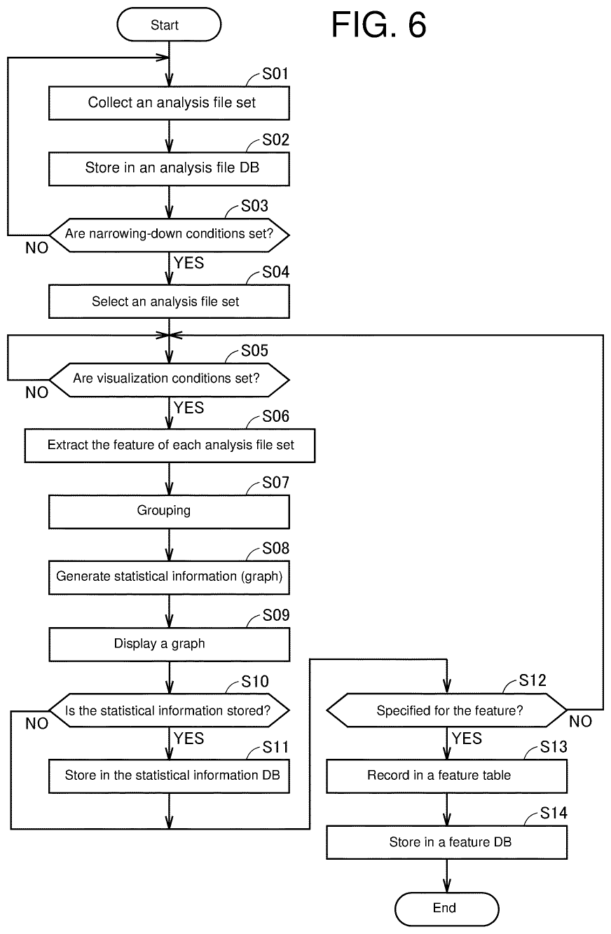

FIG. 6 is a flowchart for explaining processing procedures performed by the data processing apparatus 1.

As shown in FIG. 6, first, in Step (hereafter simply “S”) 01, the data processing apparatus 1 collects analysis file sets transmitted from the information processing apparatuses 6 of the plurality of analyzers 4 via the communication I/F 13.

In S02, the data processing apparatus 1 stores the plurality of collected analysis file sets in the analysis file DB 15A (see FIG. 4). In S02, the data processing apparatus 1 can store the sample information (sample identification information and recipe data) and the sample physical property data in association with each analysis file set.

In S03, the data processing apparatus 1 determines whether the narrowing-down conditions have been set based on the user's input operation to the operation unit 3. In the scene of generating a feature table (see FIG. 5) to be used for a statistical analysis or an AI analysis, the user can set the narrowing-down conditions for narrowing down the analysis file sets to be targeted for analysis by operating the UI screen displayed on the display unit 2 using the operation unit 3.

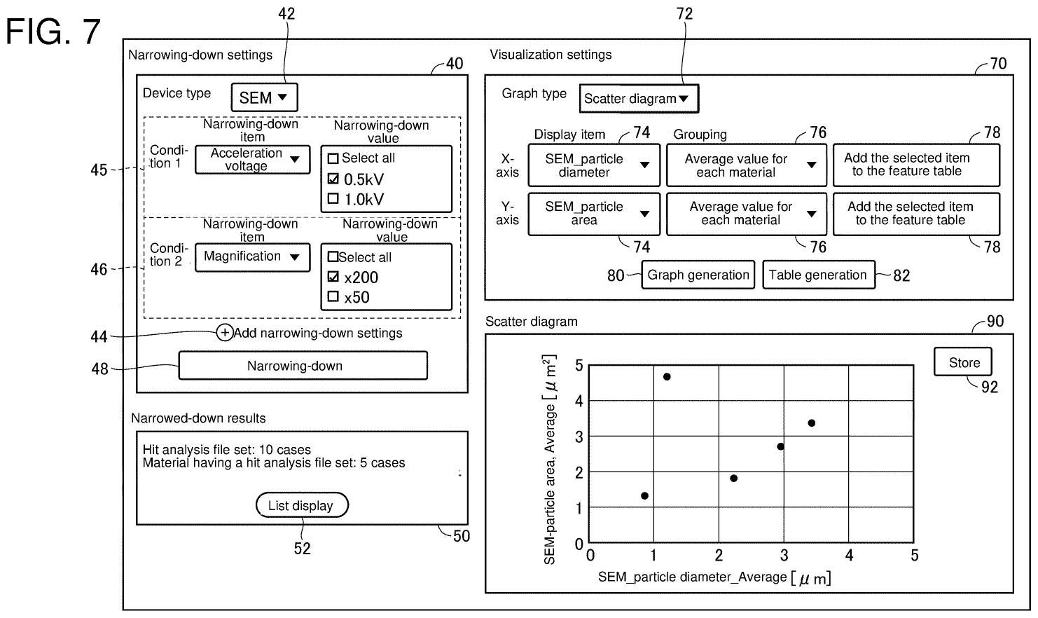

FIG. 7 is a view schematically showing a first example of a UI screen displayed on the display unit 2. As shown in FIG. 7, the UI screen is configured to include a GUI (Graphical User Interface) 40 for setting narrowing-down conditions, a GUI 50 for displaying the narrowed-down results, a GUI 70 for setting visualization conditions, and a GUI 90 for displaying a graph.

In S03 of FIG. 6, the user can set the narrowing-down conditions by operating the GUI 40 using the operation unit 3. Specifically, the GUI 40 has an icon 42 for setting the “type of the analyzer (device type).” When the user clicks on the icon 42 using the operation unit 3, a GUI (not shown) is displayed below the icon 42 showing candidate types of analyzers. The GUI lists the types of the plurality of analyzers 4 shown in FIG. 1. When the user selects one analyzer type from the plurality of types of analyzers in the list, the selected analyzer type is indicated in the GUI 42. In the example in FIG. 7, “SEM” is selected as the device type.

The GUI 40 is provided with an icon 44 for setting additional narrowing-down conditions. The user can further add narrowing-down conditions by clicking on the icon 44. In the example shown in FIG. 7, an icon 45 for setting “Condition 1” and an icon 46 for setting “Condition 2” are shown in response to the click on the icon 44.

Each of the icons 45 and 46 includes an icon for setting the “narrowing-down item” and an icon for setting the “narrowing-down value.” Although not illustrated in the figure, when the icon for a narrowing-down item is clicked, a list of items corresponding to the device type selected by the icon 42 is displayed below the icon. For example, in the case where the device type is “SEM,” the “Acceleration voltage,” “Magnification,” etc., which are measurement conditions of an SEM image, are listed in the narrowing-down items.

When the user selects a narrowing-down item from the list, the icon of the narrowing-down value located to the right shows a candidate value corresponding to the selected narrowing-down item. For example, when “Acceleration Voltage” is selected as a narrowing-down item in the icon 45 for Condition 1, the value of the acceleration voltage when the SEM image was acquired is listed as the narrowing-down value. This list can be generated by referring to the measurement conditions included in the plurality of analysis file sets including SEM images, which are saved in the analysis file DB 15A.

Note that the list includes a “Select all” item for selecting all acceleration voltage values. The user can set a narrowed-down value for the acceleration voltage by checking one of the checkboxes provided to each narrowing-down item in the list.

Similarly, in the icon 46 for Condition 2, when “Magnification” is selected as the narrowing-down item, the magnification value when the SEM image was acquired is listed in the narrowing-down value icon. The list includes a “Select all” item to select all magnification values. The user can set a narrowing-down value for the magnification by checking one of the checkboxes associated with each narrowing-down item in the list.

In this way, the user can set narrowing-down conditions for narrowing down the analysis file sets to be targeted for a statistical analysis or an AI analysis, etc., using the GUI 40. Then, by clicking on the icon 48 to execute the “narrowing down,” included in the GUI 40, the user can instruct the data processing apparatus 1 to execute the narrowing down.

Returning to FIG. 6, when the narrowing-down conditions are set, and the execution of narrowing-down is instructed (YES in S03), the data processing apparatus 1 selects an analysis file set that meets the narrowing-down conditions from a wide variety of analysis file sets saved in the analysis file DB 15A, in S04. In the example shown in FIG. 7, an analysis file set including SEM image data at an acceleration voltage of 0.5 kV and a magnification of 200× is selected.

In the GUI screen shown in FIG. 7, the GUI 50 shows the results of the narrowed-down results. Specifically, in the GUI 50, the “number of hit analysis file sets” and the “number of materials with hit analysis file sets” are shown. The number of hit analysis file sets means the number of analysis file sets that meets the narrowing-down conditions.

As shown in FIG. 4, in the analysis file DB 15A, the plurality of analysis file sets is sorted and saved by material. Therefore, the “number of materials with hit analysis file sets” means the number of materials to which such analysis file sets belong. In the example shown in FIG. 4, in the case where the analysis file set that meets the narrowing-down conditions is an analysis file set acquired from the SEM, the total number of the materials “a” to “e” to which the analysis file set belong is the number of materials with the hit analysis file set. In FIG. 7, the GUI 50 indicates that there are 10 cases of hit analysis file sets and 5 cases of materials with hit analysis file sets.

Note that the GUI 50 has an icon 52 for displaying the list of hit analysis file sets. When the user clicks on the icon 52 using the operation unit 3, information on ten (10) cases of analysis file sets and five (5) cases of materials are displayed on the UI screen. This allows the user to change and/or add narrowing-down conditions while referring to the displayed information.

Next, in S05 shown in FIG. 6, the data processing apparatus 1 determines whether a visualization condition has been set based on the user's input operation to the operation unit 3. As described above, the visualization conditions include conditions for grouping the plurality of features extracted from the plurality of analysis file sets selected in S04 and conditions for graphing the distribution state of the grouped plurality of features.

In S05, the user can set visualization conditions by operating the GUI 70 using the operation unit 3 on the UI screen shown in FIG. 7. Specifically, the GUI 70 has an icon 72 for setting the type of “graph.” First, when the user clicks on the icon 72 using the operation unit 3, a GUI (not shown) is displayed below the icon 72 showing candidates for the graph type. In this GUI, the graph type is displayed in a list. This graph expresses the distribution and/or the correlation of the plurality of data and includes, for example, a scatter diagram, a histogram, and a box-and-whisker plot. The graphs can include, in addition to the above-described statistical graphs, a graph representing analysis data itself, such as, e.g., a signal waveform diagram.

When the user selects one of the plurality of types of graphs in the list, the selected graph type is shown in the GUI 72. In the example shown in FIG. 7, “Scatter diagram” is selected as the graph type.

In the GUI 70, depending on the graph type selected by the icon 72, an icon 74 for setting items (coordinate axes) to be displayed in the graph and an icon 76 for setting grouping conditions regarding the display items (coordinate axes) are shown. For example, in the case where “Scatter diagram” is selected, an icon 74 for setting the “display items” to be displayed on the horizontal axis (X-axis) and the vertical axis (Y-axis) of the scatter diagram, and an icon 76 for setting the conditions for grouping are shown.

In the icon 74, the type of feature can be set as the display item for the graph. When the user clicks on the icon 74 using the operation unit 3, a GUI (not illustrated) indicating the candidate types of features is displayed below the icon 74. In the GUI, the types of features that can be extracted from the plurality of analysis file sets selected according to the narrowing-down conditions are listed. In the case where the selected analysis file set includes an SEM image, as shown in FIG. 7, the list includes the particle diameter, the particle area, and the average inter-particle distance of the sample acquired by analyzing the SEM image. In the example shown in FIG. 7, the X-axis display item is set to “SEM_particle diameter” and the Y-axis display item is set to “SEM_particle area.”

Once the type of the feature is set, the user can then set how the plurality of features extracted from the plurality of analysis file sets will be summarized using the icon 76. Specifically, with the icon 76, conditions for grouping the plurality of features can be set. Here, it is possible to set that the plurality of features is classified by material or by analysis file set.

Note that it is possible to choose not to perform grouping. Further, it may be possible to set the type of the statistic, which is a numerical value that summarizes the characteristics of the features classified in each group. As described above, the statistics include, for example, an average value, a median value, a mode value, a maximum value, a minimum value, a standard deviation, a variance, a skewness, and a kurtosis. In the example shown in FIG. 7, the “average value for each material” is set for each of “SEM_particle diameter” and “SEM_particle area.”

Returning to FIG. 6, when the visualization conditions are set (YES in S05), the data processing apparatus 1 performs statistical processing of the plurality of analysis file sets according to the set visualization conditions. First, in S06, the data processing apparatus 1 generates features by analyzing each analysis file set using dedicated data analysis software. In the example shown in FIG. 7, the data processing apparatus 1 analyzes the SEM image for each analysis file set and calculates the particle size and the particle area.

Next, in S07, the data processing apparatus 1 performs grouping of the plurality of features extracted from the plurality of analysis file sets. In S07, the plurality of features is classified for each material or analysis file set, according to the grouping conditions set by the icon 76 in the GUI 70, and the statistic of at least one feature classified in each group is calculated. For example, in the case where the plurality of features is classified for each material, the statistics of the features for each material are calculated.

Further, in the case where the plurality of features is classified for each analysis file set, the statistics of the features for each analysis file set are calculated. In the example shown in FIG. 7, the average SEM_particle diameter for each material and the average SEM_particle area for each material will be calculated in response to the setting of “Average value for each material” by the icon 76.

Subsequently, in S08, the data processing apparatus 1 graphs the distribution state of the plurality of features grouped in S07. In S08, the user can instruct the data processing apparatus 1 to generate a graph by clicking on the icon 80 for instructing “Graph generation” provided in the GUI 70. Upon receiving this instruction, the data processing apparatus 1 generates a graph representing the statistical distribution state of the features of the plurality of groups. In the example shown in FIG. 7, “Scatter diagram” is set as the graph type, so the data processing apparatus 1 generates a scatter diagram with the average value of the SEM_particle diameter for each material as the X-axis and the average value of the SEM_particle area for each material as the Y-axis.

In S08, the user can instruct the system to generate a table showing the distribution state of the plurality of features instead of a graph. Specifically, the user can instruct to generate a table by clicking on the icon 82 to instruct “Table generation” arranged adjacent to the icon 80. In this case, the data processing apparatus 1 generates a table describing the average value of the SEM_particle diameter and the average value of the SEM_particle area for each material.

In S09, the data processing apparatus 1 displays the generated graph on the UI screen. As shown in FIG. 7, the GUI 90 displays the generated graph (scatter diagram). In the scatter diagram, points are plotted to show the relation between the average value of the SEM_particle diameter and the average value of the SEM_particle area for each material. In the case where there are five materials with hit analysis file sets, a total of five points will be plotted on the scatter diagram.

The user can know the distribution state of the statistics of features of the plurality of groups from the graph displayed on the GUI 90. In the example shown in FIG. 7, the user can know from the scatter diagram whether there is a correlation between the average value of the SEM_particle diameter and the average value of the SEM_particle area for each material.

Further, the user can find a singularity that exists outside of the other clusters of points from the scatter diagram. There is a possibility that this singularity is due to a deficiency in at least one of the average value of the SEM_particle diameter and the average value of the SEM_particle area, which are original data,

For example, in a case where SEM image data with a measurement error is included among the plurality of SEM image data for a single material, the plurality of features calculated from the plurality of SEM image data may include outliers. As a result, there is a possibility that the average value of the plurality of features may appear as a singularity in the graph. Alternatively, in the case where the SEM image data itself is free from defects, it is conceivable that the material corresponding to the singularity possesses properties distinct from those of other materials.

However, in the case where at least one of the average value of the SEM_particle diameter and the average value of the SEM_particle area is defective, as in the former case, there is concern that recording them in the feature table may cause a problem in the later statistical analysis or AI analysis.

Therefore, in such a case, the user can choose from the following two options. As a first option, the user can change the grouping conditions to generate a graph again. For example, the currently set “average value for each material” can be changed to a “median value for each material,” a “mode for each material,” etc. In other words, the type of statistics that summarize the features of each group can be changed. When the user changes the grouping conditions and clicks on the icon 80, the GUI 90 displays a graph showing the distribution state of the statistics of the features after the change.

As a second option, the user can generate the graph again by excluding an analysis file set determined to be defective from the hit analysis file sets.

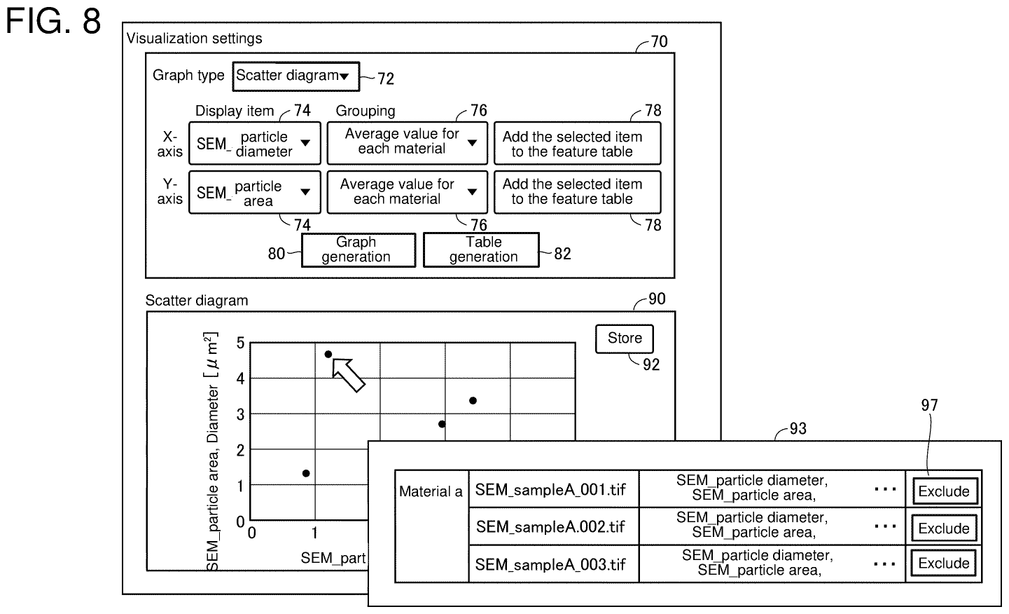

Specifically, for each point in the graph displayed in the GUI 90, the user can confirm the details of the analysis file set from which the point is originated. FIG. 8 shows the GUIs 70 and 90 extracted from the UI screen shown in FIG. 7. The GUI 90 shows a scatter diagram with the average SEM_particle diameter for each material on the X-axis and the average SEM_particle area for each material on the Y-axis. The user can select one of the five points plotted on the scatter diagram that the user would like to see the details of the original data, using the operation unit 3. When any one of the five points is selected, a GUI 93 showing the contents of the at least one analysis file set from which this one point is originated is displayed on the UI screen. In the example shown in FIG. 8, the GUI 93 includes a table showing the contents of at least one analysis file set.

The table shows the information on the material corresponding to the above-described one point and the analysis file set having this material. Each analysis file set shows the data of the SEM_particle diameter and the SEM_particle area calculated from the SEM image data included in the analysis file set. The user can confirm whether each piece of data is defective by referring to the data in each analysis file set.

The GUI 93 is provided with an icon 97 to “exclude” the data from the analysis target for each analysis file set. In the case where at least one of the SEM_particle diameter and the SEM_particle area is determined to be defective, the user can exclude this analysis file set from the analysis target by clicking on the icon 97 corresponding to the analysis file set including the data. In response to this user operation, the data processing apparatus 1 excludes the analysis file set specified by the user from the analysis file sets that hit the narrowing-down conditions.

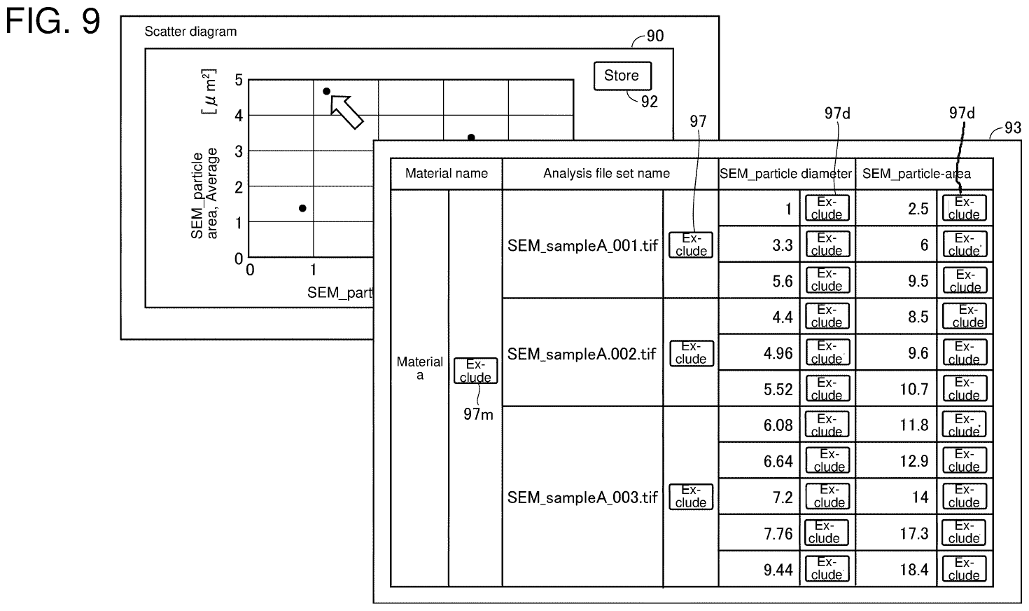

Alternatively, as shown in FIG. 9, an icon 97d can be provided to instruct the user to “exclude” data on a per-data basis. In the case where either one of the SEM_particle diameter or the SEM_particle area is determined to be deficient, the user can exclude this data from the analysis target by clicking on the icon 97d corresponding to the data. This data may be an outlier, for example, due to a measurement error. In response to this user operation, the data processing apparatus 1 excludes the data specified by the user from the analysis file sets that hit the narrowing-down conditions.

Note that the user can also exclude the materials themselves owned by these analysis file sets from the analysis targets by clicking on the icon 97m corresponding to all the analysis file sets shown in the GUI 93. In this case, in response to the user operation, the data processing apparatus 1 excludes the analysis file sets having the materials specified by the user from the analysis file sets that hit the narrowing-down conditions.

Once at least one analysis file set, data, and a material is excluded in this way, the user can instruct the data processing apparatus 1 to generate a graph based on the remaining analysis file sets by clicking the icon 80 again.

The data processing apparatus 1 performs grouping of the plurality of features extracted from the remaining analysis file sets according to the user instruction and also calculates statistics of the features classified into each group. In the examples shown in FIG. 8 and FIG. 9, the data processing apparatus 1 calculates the average value of the SEM_particle diameter for each material and the average value of the SEM_particle area for each material from the SEM_particle diameter and the SEM_particle area that are extracted from the remaining analysis file sets. The data processing apparatus 1 again generates a scatter diagram with the average value of the SEM_particle diameter for each material as the X-axis and the average value of the SEM_particle area for each material as the Y-axis, and displays it on the GUI 90.

Next, in S10, the data processing apparatus 1 determines whether or not to store the statistical information based on the user's input operation to the operation unit 3. The statistical information includes the data on the graph (or table) generated in S08. In the example shown in FIG. 7, the GUI 90 is provided with an icon 92 to instruct “Save.” When the icon 92 is clicked, in S10, it is determined to be YES. In this case, in S11, the data processing apparatus 1 stores the statistical information in the statistical information DB 15B. By storing the statistical information, after the generation of the feature table, it becomes possible for the user to check how the plurality of features recorded in the feature table were distributed.

In S12, the data processing apparatus 1 determines whether the statistics of the features of each group are specified to the features based on the user's input operation to the operation unit 3. In the case where the statistics of the features of each group are determined to be appropriate for use in the analysis from the graphs displayed in the GUI 90, the user can record the statistics in the feature table (see FIG. 5) as the features.

Specifically, in the UI screen shown in FIG. 7, the GUI 70 is provided with an icon 78 for instructing to add the statistics of the features of each group to the feature table. This icon 78 is arranged alongside the icon 76 for setting the grouping conditions for the features. In the case where the user clicks on the icon 78 using the operation unit 3, in S12, it is determined to be YES.

In the case where the statistics of the features of each group are specified as features (YES in S12), the data processing apparatus 1 records the statistics of the features of each group in the feature table in S13. For example, in response to the clicking of the icon 78 on the UI screen shown in FIG. 7, the average value of the SEM_particle diameter for each material is recorded in the column of the SEM_particle diameter for each material in the feature table (see FIG. 5), and the average value of the SEM_particle area for each material is recorded in the SEM_particle area for each material.

Note that in the case where at least one analysis file set is excluded via the GUI 93 in S09, the average value of the SEM_particle diameter for each material and the average value of the SEM_particle area for each material, which were calculated from the remaining analysis file sets, will be recorded in the feature table.

In S14, the data processing apparatus 1 stores the generated feature table in the feature DB 15C. The stored feature table is used for a statistical analysis or an AI analysis.

### Effects of this Embodiment

As described above, the data processing apparatus 1 of this embodiment is configured to provide a user interface for supporting the task of summarizing a plurality of features acquired from a plurality of analysis file sets in a situation where a feature table to be used for analysis is generated from a plurality of analysis file sets that have been selected for a target of statistical analysis or AI analysis.

Specifically, the data processing apparatus 1 classifies a plurality of features into a plurality of groups according to the grouping conditions received from the user and calculates statistics summarizing the features belonging to each group. Then, the data processing apparatus 1 presents the distribution state of the plurality of calculated statistics to the user in a graphical form.

With this, the user can visually grasp the distribution state of the plurality of statistics from the graph and, therefore, can easily determine whether the plurality of statistics is appropriate for use in the analysis. In the case where it is determined that the plurality of statistics is appropriate for use in analysis, the user can then add the plurality of statistics to the feature table by using the user interface.

On the other hand, in the case where a plurality of statistics is determined to include singularities and are not appropriate for use in analysis, the user can consider other statistics by changing the grouping conditions given to the data processing apparatus 1.

Alternatively, the user can confirm the details of the analysis file set from which the above singularity originates by using the user interface. In the event that the data is found to be defective, the user can then exclude the data, the analysis file set including the data, or the material including the analysis file set from the analysis target via the user interface.

In the case where data, analysis file sets or materials are excluded, the data processing apparatus 1 again calculates the statistics of the plurality of features from the remaining analysis file sets and presents the distribution state of the plurality of calculated statistics to the user in a graphical form. The user can determine from the graphs presented again whether the plurality of statistics is now suitable. In this manner, the user can easily acquire the statistics appropriate for analysis by using the user interface.

Further, the data processing apparatus 1 according to this embodiment is configured to store the statistical information indicating the distribution state of the plurality of statistics in accordance with the user's instruction. This allows the user to confirm how the plurality of features recorded in the feature table was distributed after generating the feature table.

### <Configuration Example of UI Screen>

Hereinafter, other configuration examples of the UI screen displayed on the display unit 2 will be described.

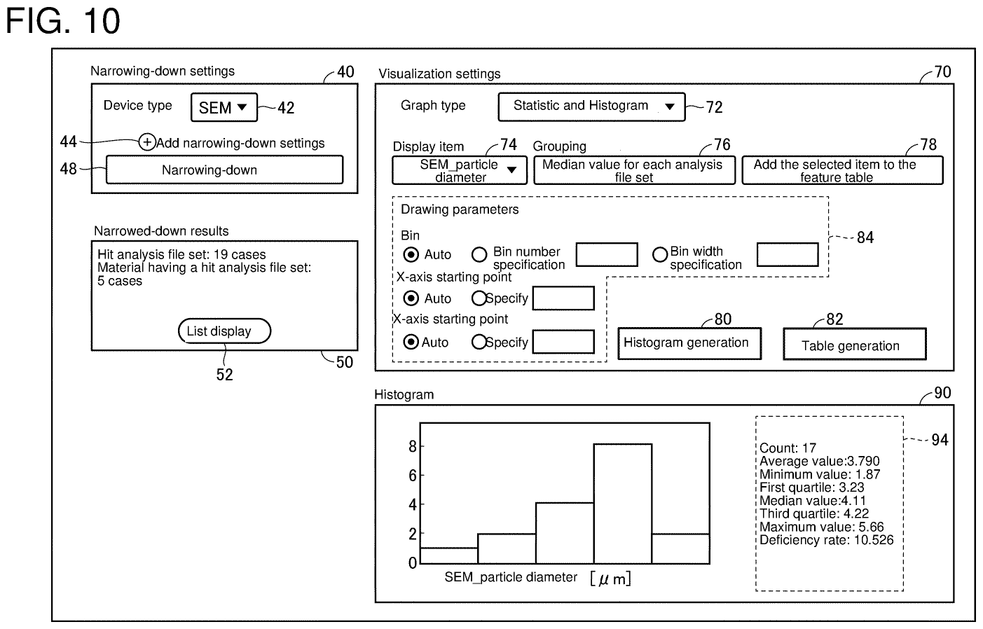

FIG. 10 is a diagram schematically showing a second example of a UI screen displayed on the display unit 2. The UI screen shown in FIG. 10 differs from the UI screen shown in FIG. 7 in the narrowing-down conditions and visualization conditions. Specifically, in the UI screen shown in FIG. 10, “SEM” is selected as the device type in the GUI 40, but no additional conditions are set. Therefore, when the data processing apparatus 1 is instructed to execute the narrowing down, it selects an analysis file set including the SEM image data from a wide variety of analysis file sets saved in the analysis file DB 15A.

The GUI 40 shows the narrowed-down results. In FIG. 10, the GUI 50 indicates that there are 19 cases of hit analysis file sets and 5 cases of materials having hit analysis file sets.

In the GUI 70, the graph type, the display item, and the grouping conditions are set as visualization conditions. The icon 72 for setting the graph type indicates that “Statistic and Histogram” is set as the graph type. The icon 74 for setting the display item indicates that “SEM_particle diameter” is set as the type of feature. The icon 76 for setting the grouping conditions indicates that “Median value for each analysis file set” has been set. Thus, in the example shown in FIG. 10, for each analysis file set, the SEM image would be analyzed and the median value of the SEM_particle diameter would be calculated. And a histogram representing the frequency distribution of the median values of the plurality of SEM_particle diameters acquired from each of the plurality of analysis file sets will be generated.

In the case where a histogram is set in the icon 72, an icon 84 for setting the parameters for drawing the histogram will appear on the GUI 70. The drawing parameters include, for example, the number and width of bins (bars) in the histogram, and the numerical range (the starting point, the ending point) of the horizontal axis (“X-axis”).

In response to the clicking of the icon 80 for instructing the generation of a histogram, the data processing apparatus 1 generates a histogram based on the drawing parameters set by the icon 84. The data processing apparatus 1 classifies the median values of the SEM_particle diameter for each analysis file set into classes equal to the number of bins and calculates the frequencies for each class. Then, a histogram is generated by plotting the frequencies of each class on the vertical axis with the horizontal axis (X-axis) as a SEM_particle diameter.

The GUI 90 displays the generated histogram. In the case where there are 19 hit analysis file sets, a histogram representing the frequency distribution of the median values of the SEM_particle diameters for a total of 19 points will be displayed. The GUI 90 further shows statistics 94 representing the distribution of median values of the SEM_particle diameters of a total of 19 points. The statistics 94 include the number of points (counts) of valid data used in the histogram, the average value, the minimum value, the first quartile value, the median value, the third quartile value, and the maximum value of the valid data, and the deficiency rate, which indicate the ratio of the number of invalid data points to the total data points.

The user can know the distribution of median values of the SEM_particle diameters of the plurality of analysis file sets from the histogram and the statistics 94 displayed on the GUI 70. The user can also learn the ratio of the invalid data from the deficiency rate included in the statistics 94.

In the case where it is determined that the distribution of the median values of the SEM_particle diameters is not appropriate or the deficiency rate is high, the user can change the grouping conditions using the icon 76 to generate the histogram again. For example, the currently set “Median value for each analysis file set” can be changed to “Mean value for each analysis file set” or “Mode value for each analysis file set” and so on. When the user changes the grouping conditions and clicks on the icon 80, the GUI 90 displays a histogram showing the frequency distribution of the statistics of the changed features.

When the icon 78 is clicked, the data processing apparatus 1 records the statistics of the features of each group in the feature table. For example, when the icon 78 is clicked on the UI screen shown in FIG. 8, the median value of the SEM_particle diameters for each analysis file set is recorded in the column of the SEM_particle diameter for each analysis file set in the feature table.

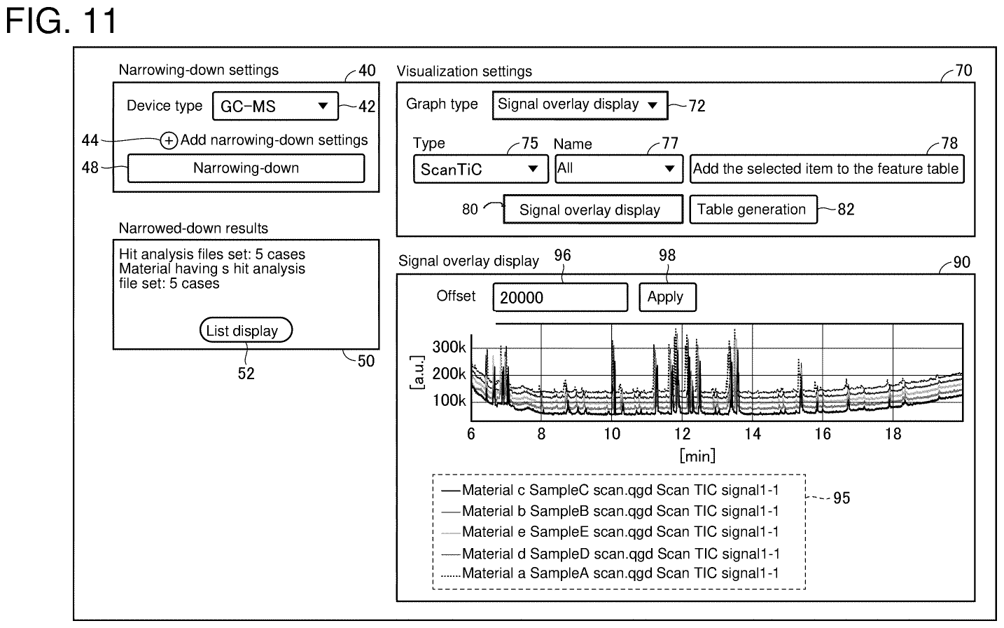

FIG. 11 is a diagram schematically showing a third example of a UI screen displayed on the display unit 2. The UI screen shown in FIG. 11 differs from the UI screen shown in FIG. 7 in the narrowing-down conditions and visualization conditions. Specifically, in the UI screen shown in FIG. 11, “GC-MS” is selected as the device type in the GUI 40. Therefore, when execution of narrowing down is instructed, the data processing apparatus 1 selects, from the wide variety of analysis file sets saved in the analysis file DB 15A, an analysis file set including the analysis data (chromatogram, mass spectrum, etc.) acquired by the GC-MS.

The GUI 50 shows the narrowed-down results. In FIG. 11, the GUI 50 indicates that there are 5 cases of hit analysis file sets and 5 cases of materials having hit analysis file sets.

In the GUI 70, the graph type, the display item, and the grouping conditions are set as visualization conditions. The icon 72 for setting the graph type indicates that “Signal overlay display” is set as the graph type. The term “Signal overlay display” means that the waveform data of the signals included in each analysis file set are displayed in a superimposed manner in a single graph.

In the case where “Signal overlay display” is set as the graph type, an icon 75 for setting the type of signal to be displayed and an icon 77 for setting the name of the signal to be displayed appear in the GUI 70.

The icon 75 allows the user to set the type of signal. When the user clicks on the icon 75 using the operation unit 3, a GUI (not shown) indicating the candidates of signal types is displayed below the icon 55 (correctly 75). The GUI displays a list of signal types included in the plurality of analysis file sets selected according to the narrowing-down conditions.

In the case where the selected analysis file set includes waveform data of a plurality of signals acquired from the GC-MS, as shown in FIG. 11, the list shows the type of the plurality of signals. In the example shown in FIG. 11, “ScanTiC” is set as the signal type. “ScanTiC” is a total ion chromatogram acquired by a scan mode analysis.

Once the signal type is set, the user can then use the icon 77 to set how the plurality of ScanTiC waveform data extracted from the plurality of analysis file sets will be summarized. For example, the icon 77 allows the user to set the number of waveform data to be displayed per analysis file set. When set to “All” as shown in FIG. 11, all ScanTiC waveform data included in each analysis file set can be displayed.

Although illustrations are omitted, in the case where “mass chromatogram” is set in the icon 75, the user can use the icon 77 to set the mass (m/z).

In response to the clicking of the icon 80 for instructing the execution of the signal overlay display, the data processing apparatus 1 generates a graph in which the waveform data of a plurality of signals corresponding to the visualization conditions are displayed in a superimposed manner on the GUI 90. The GUI 90 is provided with an icon 96 for setting the offset amount for shifting of plurality of waveform data to be displayed. The user can adjust the display method of the graph by setting an appropriate numerical value using the icon 96 and clicking on the icon 98 marked “Apply.”

In the example shown in FIG. 11, one analysis file set belongs to each of the five materials, and each analysis file set includes one piece of ScanTiC waveform data. For this reason, a total of five waveform data are superimposed on the graph. At the bottom of the graph, the text information 95 indicating the contents of each waveform data is displayed.

When the icon 78 is clicked on the GUI 70, the plurality of waveform data displayed in the graph is recorded in the feature table. In the example shown in FIG. 11, one piece of ScanTiC waveform data for each material is recorded in the feature table.

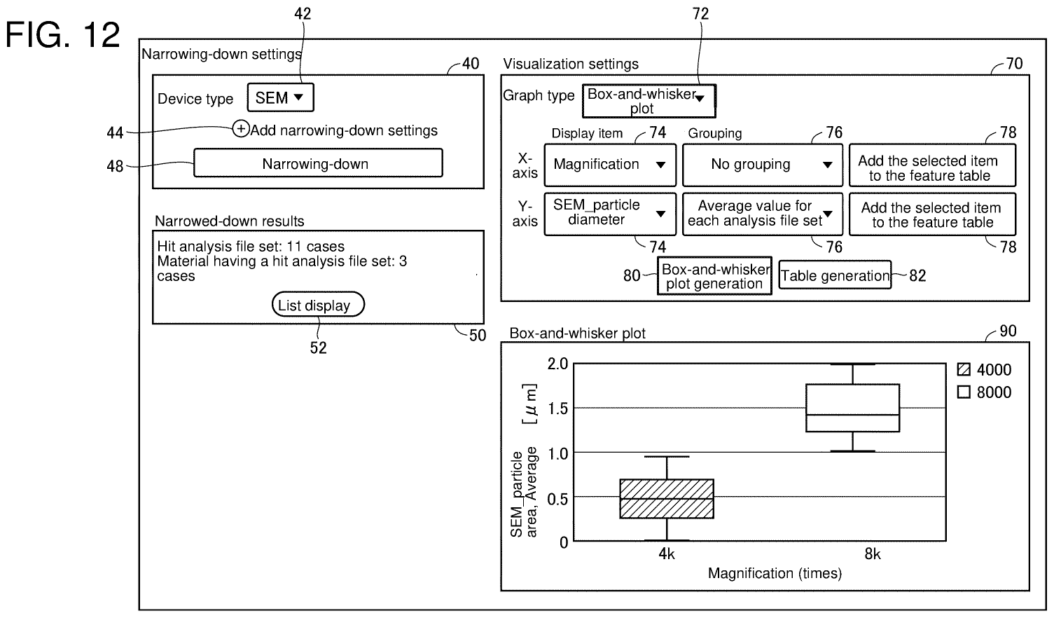

FIG. 12 is a diagram schematically showing a fourth example of a UI screen displayed on the display unit 2. The UI screen shown in FIG. 12 differs from the UI screen shown in FIG. 7 in the narrowing-down conditions and visualization conditions. Specifically, in the UI screen shown in FIG. 12, “SEM” is selected as the device type in the GUI 40, but no additional conditions are set. Therefore, when the data processing apparatus 1 is instructed to execute the narrowing down, it selects an analysis file set including the SEM image data from a wide variety of analysis file sets saved in the analysis file DB 15A.

The GUI 50 shows the narrowed-down results. In FIG. 12, the GUI 50 indicates that there are 11 cases of hit analysis file sets and 3 cases of materials having hit analysis file sets.

In the GUI 70, the graph type, the display item, and the grouping conditions are set as visualization conditions. The icon 72 for setting the graph type indicates that “Box-and-whisker plot” is set as the graph type. The icon 74 for setting the display items indicates that “(SEM image) magnification” is set on the horizontal axis (X-axis) of the box-and-whisker plot, and the “SEM_particle diameter” is set as the feature type on the vertical axis (Y-axis) of the box-and-whisker plot.

The icon 76 for setting the grouping conditions indicates that “No grouping” is set for the magnification of the SEM image. This means that the magnification of each SEM image is used as it is without summarizing the magnification of the SEM image.

The icon 76 corresponding to the SEM_particle diameter indicates that the “Average value for each analysis file set” is set. According to this, for each analysis file set, an SEM image is analyzed to calculate the average values of the SEM_particle diameters.

When the icon 80 for instructing generation of a box-and-whisker plot is clicked, the data processing apparatus 1 generates a box-and-whisker plot according to the visualization conditions. Specifically, the data processing apparatus 1 calculates the average value of the SEM_particle diameters for each analysis file set. The data processing apparatus 1 then classifies the calculated average values of the plurality of SEM_particle diameters by magnification of the SEM image and generates a box-and-whisker plot showing the distribution of the average values of the SEM_particle diameters for each magnification.

The GUI 90 displays the generated box-and-whisker plot. FIG. 12 presumes that the eleven hit analysis file sets are composed of an analysis file set including electron microscope images at a magnification of 4,000× and an analysis file set including electron microscope images at a magnification of 8,000×. In this case, the data processing apparatus 1 generates box-and-whisker plots for each of the magnifications of 4,000× and 8,000× and displays the two generated box-and-whisker plots side by side.

The user can confirm from each box-and-whisker plot the variation in the maximum value, the minimum value, and the median value of the average values of the SEM_particle diameters. Further, by comparing the two box-and-whisker plots, the variation of the average values of the SEM_particle diameters at the magnification of the SEM image can be compared.

In the case where it is determined that the variation is large in at least one of the box-and-whisker plots, the user can change the grouping conditions using the icon 76 to generate the box-and-whisker plot again. For example, the currently set “Average value for each analysis file set” can be changed to “Median value for each analysis file set” or “Mode value for each analysis file set” and so on. When the user changes the grouping conditions and clicks the icon 80, the GUI 90 displays a box-and-whisker plot showing the distribution of the statistics of the features after the change.

In response to clicking the icon 78, the data processing apparatus 1 records the statistics of the features of each group in the feature table. For example, in the case where the icon 78 is clicked on the UI screen shown in FIG. 12, the average value of the SEM_particle diameters for each analysis file set is recorded in the column of the SEM_particle diameter for each analysis file set in the feature table.

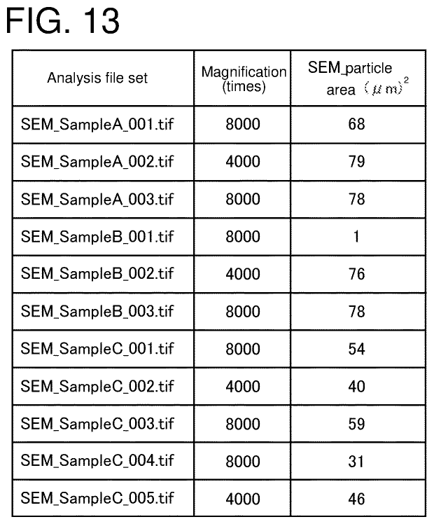

Further, when the icon 82 is clicked, the data processing apparatus 1 generates a table in which the magnification of the electron microscope image and the average value of the SEM_particle diameters are listed for each analysis file set, as shown in FIG. 13.

In the second through fourth examples of the UI screen, as in the first example, the user can determine whether the plurality of statistics is appropriate for use in analysis by confirming the distribution state of the statistics summarizing the features belonging to each group from the graphs displayed on the GUI 90. In the case where it is determined that the plurality of statistics is not appropriate, the user can consider a different statistic by changing the grouping conditions to be given to the data processing apparatus 1.

Alternatively, in the case where the user reviews the details of each analysis file set via the GUI 93 (see FIG. 8 and FIG. 9) and determines that the data is defective, it is possible to exclude the data, the analysis file set including the data, or the material having the analysis file set. In this case, the data processing apparatus 1 calculates the plurality of statistics from the remaining analysis file sets again and can again display a graph based on the calculated results on the GUI 90. In this manner, the user can easily acquire the statistics appropriate for an analysis by using the user interface.

### Aspects

It would be understood by those skilled in the art that the exemplary embodiments described above are specific examples of the following aspects.

### Item 1

A data processing method according to one aspect of the present invention comprising:
a step of collecting an analysis file set including analysis data by an analyzer from a plurality of types of analyzers;
a step of selecting a plurality of analysis file sets to be analyzed from the plurality of types of collected analysis file sets according to a narrowing-down condition received from a user;
a step of extracting a plurality of features from each of the plurality of selected analysis file sets;
a step of summarizing the plurality of features by performing statistical processing of the plurality of features; and
a step of performing an analysis using the summarized features,
wherein the step of summarizing the plurality of features includes a step of presenting statistical information generated in the statistical processing to the user.

According to the data processing method as recited in the above-described Item 1, the user can determine from the statistical information presented whether the features summarized by statistical processing are appropriate for use in statistical analysis or AI analysis. Therefore, the user can summarize the plurality of features while using statistical information and easily obtain features suitable for analyses.

### Item 2

The data processing method as recited in the above-described Item 1, further comprising:
a step of receiving a visualization condition for visualizing a distribution state of the plurality of features from the user,
wherein the visualization condition includes a grouping condition for grouping the plurality of features and a graphing condition for graphing the distribution state of the plurality of grouped features,
wherein the step of summarizing the plurality of features includes a step of classifying the plurality of features into a plurality of groups according to the grouping condition and calculating a statistic of the features belonging to each group, and
wherein the step of presenting the statistical information to the user includes a step of presenting the distribution state of the plurality of calculated statistics to the user by graphing the distribution state in accordance with the graphing condition.

According to the data processing method as recited in the above-described Item 2, the user can visually grasp the distribution state of the plurality of statistics from the graph and, therefore, can easily determine whether the plurality of statistics is appropriate for use in analysis. Further, in the case where it is determined that the plurality of statistics is not appropriate, the user can consider a different statistic by changing the grouping conditions. As a result, the user can easily acquire statistics appropriate for analysis.

### Item 3

The data processing method as recited in the above-described Item 2,
wherein the step of summarizing the plurality of features further includes a step of recording the plurality of calculated statistics in a feature table according to an instruction of the user, and
wherein the step of performing the analysis includes a step of performing the analysis using the feature table.

According to the data processing method as recited in the above-described Item 3, the user can perform the analysis using the features determined to be appropriate for use in analysis.

### Item 4

The data processing method as recited in the above-described Item 2 or 3, further comprising:
a step of providing a user interface for setting the grouping condition,
wherein the user interface presents information on a type of the statistic to the user.

According to the data processing method as recited in the above-described Item 4, the user can execute statistical processing while changing grouping conditions using the user interface, thus enabling the user to efficiently acquire statistics appropriate for analysis.

### Item 5

The data processing method as recited in any one of the above-described Items 2 to 4, further comprising:
a step of providing a user interface for setting the graphing condition,
wherein the user interface presents information on a type of a graph and a coordinate axis of the graph to the user.

According to the data processing method as recited in the above-described Item 5, the user can grasp the distribution state of a plurality of statistics from multiple angles by changing the graph type and/or the coordinate axes using the user interface.

### Item 6

The data processing method as recited in the above-described Items 1 to 5, further comprising:
a step of providing a user interface for setting a feature to be extracted from the plurality of analysis file sets,
wherein the user interface presents information on a type of a feature extractable from the plurality of analysis file sets to the user.

According to the data processing method as recited in the above-described Item 6, the user can easily set the features to be extracted from the plurality of analysis file sets.

### Item 7

The data processing method as recited in any one of the above-described Items 2 to 6,
wherein the step of presenting the statistical information to the user further includes a step of presenting information on the features belonging to each group to the user according to an instruction of the user.

According to the data processing method as recited in the above-described Item 7, the user can confirm whether the features belonging to each group include outliers due to measurement errors or other reasons.

### Item 8

The data processing method as recited in any one of the above-described Items 2 to 7,
wherein the step of presenting the statistical information to the user further includes a step of excluding at least one feature from the plurality of features according to the instruction of the user, and
wherein the step of summarizing the plurality of features further includes a step of recalculating the statistics of the features belonging to each group according to the grouping condition for the plurality of features from which the at least one feature has been excluded.

According to the data processing method as recited in the above-described Item 8, the user can exclude outliers from the features belonging to each group. Alternatively, the group itself including outliers can be excluded.

### Item 9

The data processing method as recited in the above-described Item 8,
wherein the step of presenting the statistical information to the user further includes a step of presenting the distribution state of the plurality of recalculated statistics to the user by graphing the distribution state in accordance with the graphing condition.

According to the data processing method as recited in the above-described Item 9, the user can reconsider from the graph whether the remaining feature statistics are appropriate to be used in analysis.

### Item 10

The data processing method as recited in any one of the above-described Items 1 to 9, further comprising:
a step of providing a user interface for setting the narrowing-down condition,
wherein the user interface presents information on a type of an analysis file set selectable from the plurality of types of collected analysis file sets to the user.

According to the data processing method as recited in the above-described Item 10, the user can easily set the features to be extracted from the plurality of analysis file sets.

### Item 11

The data processing method as recited in one of the above-described Items 1 to 10, further comprising:
a step of storing the statistical information in a storage device.

According to the data processing method as recited in the above-described Item 11, after summarizing a plurality of features, the user can confirm how the plurality of features was distributed, based on statistical information.

### Item 12

A data processing apparatus capable of communicating with a plurality of types of analyzers, the data processing apparatus comprising:
a processor; and
a memory configured to store a program to be executed by the processor,
wherein the processor is configured to, in accordance with the program,
collect an analysis file set including analysis data by an analyzer from the plurality of types of analyzers,
select a plurality of analysis file sets to be analyzed from the plurality of types of collected analysis file sets according to a narrowing-down condition received from a user,
extract a plurality of features from each of the plurality of selected analysis file sets,
summarize the plurality of features by performing statistical processing of the plurality of features, and
perform an analysis using the summarized features,
wherein the processor presents statistical information generated in the statistical processing to the user when summarizing the plurality of features.

According to the data processing method as recited in the above-described Item 12, the user can determine from the presented statistical information whether the features summarized by statistical processing are appropriate for use in statistical analysis or AI analysis. Therefore, the user can summarize the plurality of features while using statistical information and easily obtain features suitable for analyses.

### Item 13

A non-transitory computer-readable storage medium storing a program,
wherein the program makes a computer execute:
a step of collecting an analysis file set including analysis data by an analyzer from a plurality of types of analyzers;
a step of selecting a plurality of analysis file sets to be analyzed from the plurality of types of collected analysis file sets according to a narrowing-down condition received from a user;
a step of extracting a plurality of features from each of the plurality of selected analysis file sets;
a step of summarizing the plurality of features by performing statistical processing of the plurality of features; and
a step of performing an analysis using the summarized features,
wherein the step of summarizing the plurality of features includes a step of presenting statistical information generated in the statistical processing to the user.

According to the program as recited in the above-described Item 13, the user can determine from the presented statistical information whether the features summarized by statistical processing are appropriate for use in statistical analysis or AI analysis. Therefore, the user can summarize the plurality of features while using statistical information, and can easily obtain features suitable for analyses.

Further, it should be noted that in the embodiment and modifications, it is planned from the beginning of the application to combine the configurations described in the embodiments as appropriate, including combinations not mentioned in the specification, to the extent that no inconvenience or inconsistency arises.

Although some embodiments of the present invention have been described, the embodiments disclosed here should be considered in all respects illustrative and not restrictive. It should be noted that the scope of the present invention is indicated by claims and is intended to include all modifications within the meaning and scope of the claims and equivalents.

1. A data processing method comprising:
a step of collecting an analysis file set including analysis data by an analyzer from a plurality of types of analyzers;
a step of selecting a plurality of analysis file sets to be analyzed from a plurality of types of collected analysis file sets according to a narrowing-down condition received from a user;
a step of extracting a plurality of features from each of the plurality of selected analysis file sets;
a step of summarizing the plurality of features by performing statistical processing of the plurality of features; and
a step of performing an analysis using the summarized features,
wherein the step of summarizing the plurality of features includes a step of presenting statistical information generated in the statistical processing to the user.

2. The data processing method as recited in claim 1, further comprising:
a step of receiving a visualization condition for visualizing a distribution state of the plurality of features from the user,
wherein the visualization condition includes a grouping condition for grouping the plurality of features and a graphing condition for graphing the distribution state of the plurality of grouped features,
wherein the step of summarizing the plurality of features includes a step of classifying the plurality of features into a plurality of groups according to the grouping condition and calculating a statistic of the features belonging to each group, and
wherein the step of presenting the statistical information to the user includes a step of presenting the distribution state of the plurality of calculated statistics to the user by graphing the distribution state in accordance with the graphing condition.

3. The data processing method as recited in claim 2,
wherein the step of summarizing the plurality of features further includes a step of recording the plurality of calculated statistics in a feature table according to an instruction of the user, and
wherein the step of performing the analysis includes a step of performing the analysis using the feature table.

4. The data processing method as recited in claim 2, further comprising:
a step of providing a user interface for setting the grouping condition,
wherein the user interface presents information on a type of the statistic to the user.

5. The data processing method as recited in claim 2, further comprising:
a step of providing a user interface for setting the graphing condition,
wherein the user interface presents information on a type of a graph and a coordinate axis of the graph to the user.

6. The data processing method as recited in claim 1, further comprising:
a step of providing a user interface for setting a feature to be extracted from the plurality of analysis file sets,
wherein the user interface presents information on a type of a feature extractable from the plurality of analysis file sets to the user.

7. The data processing method as recited in claim 2,
wherein the step of presenting the statistical information to the user further includes a step of presenting information on the features belonging to each group to the user according to an instruction of the user.

8. The data processing method as recited in claim 7,
wherein the step of presenting the statistical information to the user further includes a step of excluding at least one feature from the plurality of features according to the instruction of the user, and
wherein the step of summarizing the plurality of features further includes a step of recalculating the statistics of the features belonging to each group according to the grouping condition for the plurality of features from which the at least one feature has been excluded.

9. The data processing method as recited in claim 8,
wherein the step of presenting the statistical information to the user further includes a step of presenting the distribution state of the plurality of recalculated statistics to the user by graphing the distribution state in accordance with the graphing condition.

10. The data processing method as recited in claim 1, further comprising:
a step of providing a user interface for setting the narrowing-down condition,
wherein the user interface presents information on a type of an analysis file set selectable from the plurality of types of collected analysis file sets to the user.

11. The data processing method as recited in claim 1, further comprising:
a step of storing the statistical information in a storage device.

12. A data processing apparatus capable of communicating with a plurality of types of analyzers, the data processing apparatus comprising:
a processor; and
a memory configured to store a program to be executed by the processor,
wherein the processor is configured to, in accordance with the program,
collect an analysis file set including analysis data by an analyzer from the plurality of types of analyzers,
select a plurality of analysis file sets to be analyzed from the plurality of types of collected analysis file sets according to a narrowing-down condition received from a user,
extract a plurality of features from each of the plurality of selected analysis file sets,
summarize the plurality of features by performing statistical processing of the plurality of features, and
perform an analysis using the summarized features,
wherein the processor presents statistical information generated in the statistical processing to the user when summarizing the plurality of features.

13. A non-transitory computer-readable storage medium storing a program,
wherein the program makes a computer execute:
a step of collecting an analysis file set including analysis data by an analyzer from a plurality of types of analyzers;
a step of selecting a plurality of analysis file sets to be analyzed from the plurality of types of collected analysis file sets according to a narrowing-down condition received from a user;
a step of extracting a plurality of features from each of the plurality of selected analysis file sets;
a step of summarizing the plurality of features by performing statistical processing of the plurality of features; and
a step of performing an analysis using the summarized features,
wherein the step of summarizing the plurality of features includes a step of presenting statistical information generated in the statistical processing to the user.
